(19) United States
(12) Patent Application Publication
(10) Pub. No.: US 2021/0161471 A1
(43) Pub. Date: Jun. 3, 2021

(54) MULTI-LAYER STRUCTURE, SYSTEM, USE AND METHOD

(71) Applicant: Acquandas GmbH, Kiel (DE)

(72) Inventor: Rodrigo LIMA DE MIRANDA, Kiel (DE)

(21) Appl. No.: 16/772,883

(22) PCT Filed: Dec. 6, 2018
(86) PCT No.: PCT/EP2018/083737

§ 371 Date: Jun. 15, 2020

(30) Foreign Application Priority Data
Dec. 15, 2017 (DE) 10 2017 130 152.6

(51) Int. Cl. A61B 5/00; A61B 5/294; A61N 1/05; H01B 3/08; H01B 1/02; H01B 7/04

(52) U.S. Cl. CPC A61B 5/6847; A61B 5/294; A61N 1/0551; A61B 2562/028; H01B 1/02; H01B 7/048; H01B 3/08

(57) ABSTRACT

The invention relates to a multi-layer structure having at least one flexible backing layer, at least one electrically insulating layer, and at least one electrically conductive layer, the electrically insulating layer being arranged between and connected to the backing layer and the electrically conductive layer, at least the backing layer being able to be elongated by at least 0.5% and comprising a shape memory material that is adapted to transmit restoring forces to mend cracks in the electrically insulating layer.

The invention relates to a multi-layer structure, a system, the use of the multi-layer structure and a method for the self-mending of a multi-layer structure and a method for operating a multi-layer structure.

Multi-layer structures are used in microelectronics, for example, in microelectrode arrays (MEA). Such electrodes are intended to monitor and/or stimulate neural activities. Such multi-layer structures usually have a backing substrate on which conductor layers are applied, which conductor layers are separated from the backing substrate by electrically insulating layers.

Materials which have similar elasticities are used for insulated electrical connections on flexible backing substrates in order to achieve a certain flexibility of the multi-layer structure. These can be inorganic/polymeric insulation materials and metallic conducting path materials. When inorganic/oxidic insulation materials are used, alternating loads, in particular alternating loads with larger elongations, are difficult to achieve. In the prior art, polymeric/organic materials are used as thin electrical insulators for permanent, insulated-electrical connections with a flexible backing substrate. Polymers tend to age and degenerate in the biological environment, which in the medium term leads to functional defects in the insulation and conducting paths, especially under load.

A further approach is the use of alternating multi-layer thin layers (nanolaminates) made of organic/polymeric and inorganic/oxidic substrates. The layer adhesion between different organic and inorganic materials is complex and mostly unsatisfactory. Insulation defects and degeneration occur in the biological environment.

A layout of the design and the insulation layer as multiple conductors and insulators is conceivable, so that the layers do not experience great mechanical elongations under load. This has the disadvantage in that it is practically impossible to achieve a large elongation only through a mechanical design. The cross-sections become smaller and the possible signal amplitudes decrease. The electrical insulation and conductor materials can break and thus lose their electrical functional properties when connected with highly flexible substrate layers and under alternating loads when the alternating loads exceed relatively low limit values.

The invention is based on the object of specifying a multi-layer structure, in particular for microelectronic applications, which is mechanically flexible and retains its electrical properties as well as possible under load. The invention is also based on the object of specifying a system having such a multi-layer structure, the use of a multi-layer structure and a method for the self-mending of a multi-layer structure or for operating a multi-layer structure.

According to the invention, this object is achieved with a view to the multi-layer structure by the subject matter of claim 1. With a view to the system, the object is achieved by the subject matter of claim 10, with a view to the use, by the subject matter of claim 11, with a view to the method for self-mending, by the subject matter of claim 12 and with a view to the method for operating a multi-layer structure, by the subject matter of claim 14.

Specifically, the object is achieved by a multi-layer structure having at least one flexible backing layer, at least one electrically insulating layer, and at least one electrically conductive layer. The electrically insulating layer is arranged between the backing layer and the electrically conductive layer and is connected to them in each case. At least the backing layer is able to be elongated by at least 0.5% and comprises a shape memory material that is adapted to transmit restoring forces to mend cracks in the electrically insulating layer.

The invention enables self-mending, insulated-electrical connections, for example, for the transmission or detection of electrical signals, voltages or currents in, for example, bioelectronic implants. The invention therefore takes a different path than the prior art. The invention allows cracks in the electrically insulating layer itself, since these are mended again. The cracks here are closed to such an extent that the electrical properties of the multi-layer structure are impaired less overall than is the case in the prior art. A complete mending of cracks in the sense that cracks at least macroscopically completely disappear is not absolutely necessary. It is sufficient that the cracks are closed to such an extent that the original electrical properties of the multi-layer structure before the mechanical load are largely preserved.

For self-mending, it is provided that the backing layer comprises a shape memory material that is adapted to transmit restoring forces to the electrically insulating layer. The restoring forces arise in a manner known per se from the phase transformation inherent in shape memory materials. According to the invention, the backing layer is able to be elongated by at least 0.5% for its voltage-induced phase transformation. The restoring forces that occur here act between the backing layer and the electrically insulating layer and lead to any cracks formed in the electrically insulating layer being closed or largely closed. For this reason, the forces acting between the backing layer and the electrically insulating layer are referred to as restoring forces, since these forces at least largely return the electrically insulating layer locally to the initial state or to a state in which the layer is largely crack-free or at least has few cracks. The multi-layer structure according to the invention can thus be subjected to large loads or elongations without the electrical properties of the multi-layer structure being significantly impaired. In particular, elongations of more than 0.5% are possible.

Elongation is understood to mean the relative change in dimension, in particular a relative change in length (lengthening or shortening) of the backing layer or generally one layer or of the entire multi-layer structure under load. The load can be caused, for example, by a force or by a change in temperature (thermal expansion). The relative change in dimension, in particular the relative change in length, occurs mainly in the plane spanned by the respective layer. When the dimension of the body increases, one speaks of a positive elongation (extension), otherwise of a negative elongation or compression.

The elongation is defined as

$$ɛ = \frac{Δ 𝓁}{{𝓁}_{0}} ,$$

where Δl is the change in length or generally the change in dimension and l₀ is the original length or generally the original dimension.

The elongation can preferably range from 0.5% to 10% or more. In other words, the elongation can be 0.5% to 10%. The lower limit for the range of elongation is at least 0.5%, preferably at least 1 percent, at least 1.5%, at least 2%, at least 2.5%, at least 3%, at least 3.5%, at least 4%.

The layer arrangement of the individual layers in the multi-layer structure is not subject to any particular restrictions. It is only necessary for the restoring forces to be able to be transferred from the backing layer to the electrically insulating layer. For example, it is possible for a plurality of electrically insulating layers and electrically conductive layers to be arranged alternately on a single backing layer. It is also possible for the multi-layer structure to have a plurality of layer units, each comprising at least one flexible backing layer, at least one electrically insulating layer and at least one electrically conductive layer. The layer units themselves can in turn have a single backing layer on which a plurality of electrically insulating layers and electrically conductive layers are arranged alternately.

The invention enables self-mending properties of the multi-layer structure in connection with permanent or continuous insulated electrical connections as well as in connection with discretely insulated electrical connections. Electrical conductors and insulators having a flexible backing layer, such as Nitinol, can be used under mechanical loads or alternating loads with an elongation of greater than 0.5%, so that a continuous or discrete permanent transmission of electrical signals, voltages and currents is possible.

Preferred embodiments are specified in the dependent claims.

When the backing layer, the electrically insulating layer and the electrically conductive layer are able to be elongated together by at least 0.5%, the stability of the layer composite is improved. At the same time, the deformation required for the phase transformation of the backing layer is achieved.

The layer thickness of the electrically insulating layer or the electrically conductive layer can in each case be at most 50 μm. Other layer thicknesses are possible. A layer thickness of the electrically insulating layer between 1 nm and 8 μm is particularly preferred.

The layers are preferably arranged so close to one another that Van der Waals forces act between the boundary layers of the different material layers.

Reference is made to the dependent claims with regard to the preferred and possible materials for the backing layer, the electrically insulating layer and the electrically conductive layer.

In the context of the system according to the invention, a multi-layer structure according to the invention and a mechanical actuator is claimed, which actuator is connected to the multi-layer structure for operating the multi-layer structure. In other words, the mechanical actuator is provided to initiate or trigger the elongation of the multi-layer structure or at least the backing layer.

The use of the multi-layer structure according to the invention is not limited to medical applications, which form a very important application. The invention can be used in all possible technical fields in which microelectronic components are used and subjected to loads. Examples of corresponding uses are specified in claim 11.

In the context of the method according to the invention, for the self-mending of a multi-layer structure according to claim 1, said multi-layer structure is elongated by at least 0.5%. This induces the voltage required for the generation of the restoring forces, which leads to the phase transformation.

When the load on the multi-layer structure that occurs during operation, for example, an alternating load, leads to an elongation of at least 0.5% of the multi-layer structure, or at least the backing layer, an automatic self-mending of any cracks in the electrically insulating layer is achieved. The load responsible for self-mending, in particular alternating load, can be superimposed on another load that occurs during operation, so that an automatic self-mending of any cracks in the electrically insulating layer is then also brought about.

In the context of the method according to the invention, for operating a multi-layer structure according to claim 1, an electrical voltage is applied to the electrically conductive layer. The multi-layer structure is subjected to an alternating load in which the multi-layer structure is elongated by at least 0.5%. The elongation is adjusted so that a continuous current flows through the electrical line during the alternating stress or that the current through the electrical line is interrupted according to the frequency of the alternating stress during the alternating stress.

In the method according to the invention for operating the multi-layer structure according to claim 1, for example, as a microelectrode, an alternating load is impressed on the multi-layer structure permanently or at least for a longer continuous period. The alternating load leads to the restoring forces between the backing layer and the electrically insulating layer acting permanently or for a longer period, so that a continuous self-mending effect is generated.

Two different operating options or operating states are to be distinguished here. The elongation generated in connection with the alternating load can be so low (but not less than 0.5%) that a continuous, uninterrupted current flows through the electrically conductive layer. Alternatively, the alternating load can be set so high that the current flow through the electrically conductive layer is interrupted in a maximum amplitude range of the alternating load, so that the current flows discretely, that is, non-continuously, through the electrically conductive layer.

The invention is described below with reference to exemplary embodiments and with reference to the accompanying schematic drawings with further details.

These show

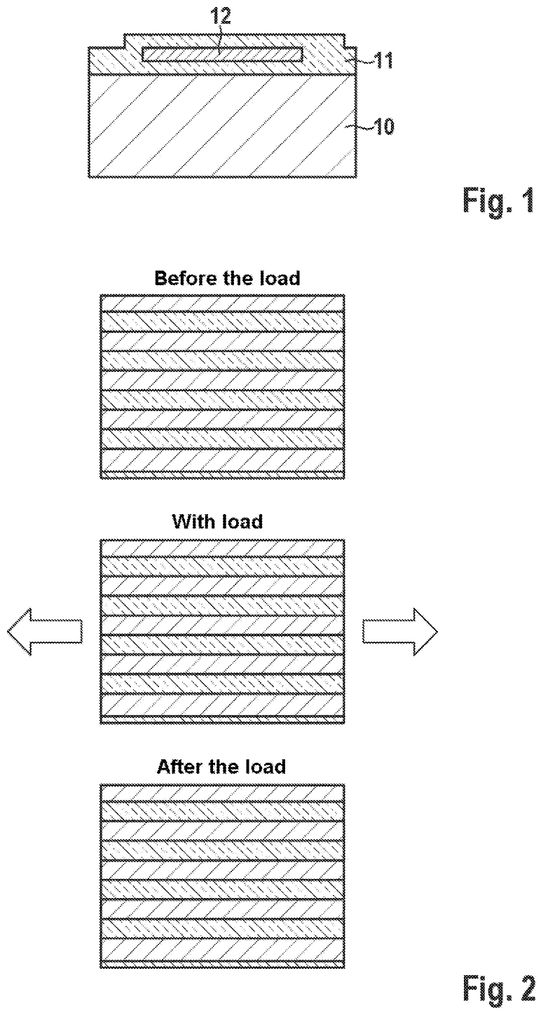

FIG. 1 a cross-section through a multi-layer structure having a backing layer, an electrically insulating layer and an electrically conductive layer according to an embodiment of the invention;

FIG. 2 a cross-section through a multi-layer structure according to an embodiment according to the invention before application of a load, during the load and after the load; and

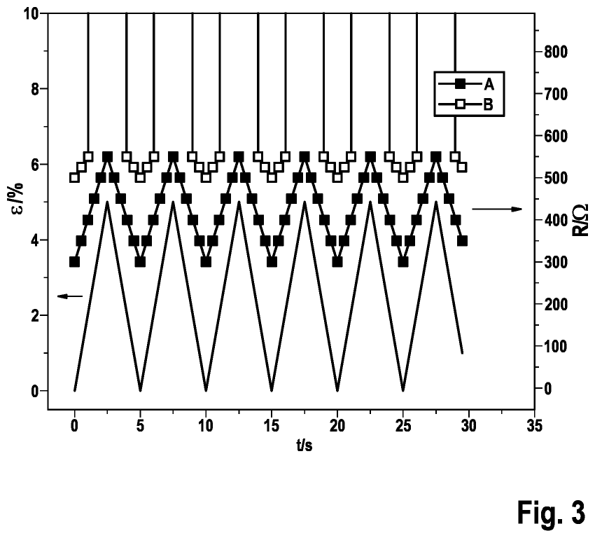

FIG. 3 a diagram showing the curve of the resistance as a function of an alternating load over time.

FIG. 1 shows a cross-section through a multi-layer structure according to an embodiment of the invention. This can be, for example, a flexible, electrically insulated connection, which can generally be referred to as a multi-layer device or as a multi-layer system. The multi-layer structure forms a central component of the multi-layer system. An example of a multi-layer system is a multi-channel connector. The multi-layer structure shown is preferably used in the medical field. Other applications are possible.

Examples of such applications are applications
in a medical, bioelectronic implant, in particular for the electrical detection and stimulation of biological tissue,
in a sensor or BioMEMS as an electrically insulated conducting path,
for the detection of biological signals,
in medical, industrial and lifestyle applications as an electrically insulated conducting path for the transmission of electrical signals, voltages or currents,
in connection plugs and connection connectors as an electrically insulated connection,
in connections to implants and wearables as an electrically insulated connection.

The multi-layer structure according to FIG. 1 is constructed in three layers. An electrically insulating layer 11 is applied to a backing layer 10. An electrically conductive layer 12 is applied to the electrically insulating layer 11. The electrically conductive layer 12 is electrically insulated from the backing layer 10 by the electrically insulating layer 11. In the example according to FIG. 1, the electrically conductive layer 12 is encased by the electrically insulating layer 11, so that both the side facing the backing layer 10 and the side of the electrically conductive layer 12 facing away from the backing layer 10 are electrically insulated.

The multi-layer structure can have a plurality of electrically insulating layers 11 and electrically conductive layers 12 in sandwich construction or alternately one above the other. The electrically conductive layer 12 forms conducting paths which are interconnected for the function of the multi-layer structure or the corresponding system.

The backing layer 10 is made from a shape memory material. A nickel-titanium alloy is used for this in the example according to FIG. 1. Other shape memory materials are possible.

The material of the backing layer can be selected, for example, from the group
Nitinol,
beta titanium,
NiTi alloys,
NiTiCu alloys,
NiTiX alloys and
polymers

without being limited to this.

The backing layer can be elongated by at least 0.5%. Specifically, the entire multi-layer structure can be elongated by 0.5%. A corresponding elongation causes a phase transformation in the backing layer which is indicated by tension, so that corresponding forces, that is, restoring forces, are transmitted from the backing layer 10 to the electrically insulating layer 11. Any cracks formed in the electrically insulating layer 11 are eliminated or mended by these forces. Complete elimination is not necessary. It suffices when the electrically insulating layer 11 has fewer cracks after loading than before loading.

In the optimal case, the electrically insulating layer 11 is free of cracks before loading. During and after the loading, any cracks are suppressed or mended by the forces generated by the backing layer 10.

The backing layer 10 is flexible.

As can be seen from FIG. 1, the layer thickness of the backing layer 10 is greater than the layer thickness of the electrically insulating layer 11 and the electrically conductive layer 12 together. Other conditions are possible. For example, the layer thickness of the electrically insulating layer 11 is 600 nm, that is, the layer thickness between the electrically conductive layer 12 and the backing layer 10 is 600 nm. The layer thickness of the electrically conductive layer is 300 nm in this exemplary embodiment. The layer thickness of the insulator on the top side or on the side of the electrically conductive layer 11 facing away from the backing layer 10 is 300 nm in the embodiment. The layer thickness of the backing layer 10 can be 30 μm, for example. Other layer thicknesses are possible.

In general, the layer thickness of the electrically insulating layer 11 can be between 1 nm and 8 μm.

Reference is made to claims 5 to 8 regarding the materials of the electrically insulating layer 11 and the electrically conductive layer 12. Other materials are possible.

FIG. 2 shows a cross-section through a multi-layer structure according to an example according to the invention. The upper illustration in FIG. 2 shows a cross-section through the individual layers before they are loaded. The middle representation shows the individual layers during the loading. The lower illustration shows the individual layers after the loading. As a result, the layers during and after loading essentially correspond to the crack-free layers before loading. There is practically no difference. This is due to the self-mending effect of the multi-layer structure according to the example according to the invention.

FIG. 3 shows, based on a diagram, two different methods for operating a multi-layer structure according to an example according to the invention, for example, in the context of one of the above uses. The method is based on the fact that the multi-layer structure is subjected to an alternating load, so that there is a continuous self-mending effect, as described above.

Method A is a permanent and continuous electrical connection. The electrical conducting path on the insulator changes the electrical resistance under alternating loads. The resistance increases with increasing elongation, and the resistance decreases with decreasing elongation of the backing substrate. However, the insulation and electrical conduction are continuously present and can be subjected to permanent loads.

Method B results in a discrete electrical connection. The electrical connection is interrupted periodically, namely at the frequency of the alternating load. When a critical elongation value is exceeded, the connection is broken. If the elongation falls below this critical value, the electrical connection is present again and continuously. These processes are practically infinitely reproducible.

Possible manufacturing processes are:
physical vapor deposition (PVD), including magnetron sputter deposition
chemical vapor deposition (CVD), including atomic layer deposition, PECVD,
thermal deposition

A shaping of the multi-layer structure by thermomechanical heat treatment is possible. This can be done, for example, by crystallization of the amorphously deposited shape memory material under mechanical load by heat treatment in a high vacuum furnace.

1. A multi-layer structure having
at least one flexible backing layer,
at least one electrically insulating layer, and
at least one electrically conductive layer,
the electrically insulating layer being arranged between and connected to the backing layer and the electrically conductive layer, at least the backing layer being able to be elongated by at least 0.5% and comprising a shape memory material which is adapted to transmit restoring forces to mend cracks in the electrically insulating layer.

2. The multi-layer structure according to claim 1, wherein
the backing layer, the electrically insulating layer and the electrically conductive layer are together able to be elongated by at least 0.5%.

3. The multi-layer structure according to claim 1, wherein
Van der Waals forces act between the boundary layers of the different material layers.

4. The multi-layer structure according to claim 1, wherein
the material of the backing layer is selected from the group
Nitinol,
beta titanium,
NiTi alloys,
NiTiCu alloys,
NiTiX alloys and
polymers.

5. The multi-layer structure according to claim 1, wherein
the material of the electrically insulating layer is selected from the group
SiO2, SiO, SiOx,
Al2O3
TiO2
NbO, NbO2, Nb2O5
TaO, TaO2, Ta2O5
ZrO2 (stabilized with (Y, Ca, Mg, Ce, Al, Hf) oxides) or from the group
AlN
TiN, 1:1 ratio may differ
Si3N4
TaN, (there are also Ta2N, Ta2N3, Ta3N5, Ta4N5, Ta5N6) or from the group
SiC.

6. The multi-layer structure according to claim 5, wherein
additions are selected from the group
Y2O3
WO2
MoO3
MoO2
ZnO
MgO
CaO
Na2O
P2O5
Fe2O3.

7. The multi-layer structure according to claim 1, wherein
the material of the electrically insulating layer comprises a bioglass, in particular having the composition 45% by weight SiO2, 24.5% by weight CaO, 24.5% by weight Na2O, and 6.0% by weight P2O5.

8. The multi-layer structure according to claim 1, wherein
the material of the electrically conductive layer is selected from the group
NiTi alloys,
PtIr alloys,
Ta and alloys thereof,
Pt and alloys thereof,
Au and alloys thereof,
Ag and alloys thereof,
polymeric materials and
carbon-containing materials.

9. The multi-layer structure according to claim 1, wherein
the layer thickness of the electrically insulating layer is between 1 nm and 8 μm.

10. A system having a multi-layer structure according to claim 1 and a mechanical actuator which is connected to the multi-layer structure for the elongation of the multi-layer structure.

11. A use of the multi-layer structure according to claim 1,
in a medical, bioelectronic implant, in particular for the electrical detection and stimulation of biological tissue,
in a sensor or BioMEMS as an electrically insulated conducting path,
for the detection of biological signals,
in medical, industrial and lifestyle applications as an electrically insulated conducting path for the transmission of electrical signals, voltages or currents,
in connection plugs and connection connectors as an electrically insulated connection,
in connections to implants and wearables as an electrically insulated connection.

12. A method for self-mending of a multi-layer structure according to claim 1, in which the multi-layer structure is elongated by at least 0.5%.

13. The method according to claim 10, wherein
the multi-layer structure is subjected to an alternating load for elongation.

14. A method for operating a multi-layer structure according to claim 1, in which an electrical voltage is applied to the electrically conductive layer and the multi-layer structure is subjected to an alternating load, in which the multi-layer structure is elongated by at least 0.5%, the elongation being adjusted such that a continuous current flows through the electrical conductor during the alternating stress or that the current through the electrical conductor is interrupted according to the frequency of the alternating stress during the alternating stress.

15. The method according to claim 11, wherein
the backing layer has a residual elongation of at most 1% after loading.
